(19) United States
(12) Patent Application Publication
(10) Pub. No.: US 2009/0123951 A1
(43) Pub. Date: May 14, 2009

(54) Method Of Diagnosing A Body Weight Condition Or Predisposition

(76) Inventors: Gene Friesen Kim, Carthage, IN (US); Ryan Michael Yamka, Topeka, KS (US); William David Schoenherr, Hoyt, KS (US); Philip William Toll, Valley Falls, KS (US); Cheryl Christine Miller, Lawrence, KS (US)

Correspondence Address: COLGATE-PALMOLIVE COMPANY, 909 RIVER ROAD, PISCATAWAY, NJ, 08855, US

(21) Appl. No.: 12/159,678

(22) PCT Filed: Nov. 30, 2006
(86) PCT No.: PCT/US06/61432

§ 371 Date: Oct. 30, 2008

Related U.S. Application Data
(60) Provisional application No. 60/754,482, filed on Dec. 28, 2005.
(60) Provisional application No. 60/819,298, filed on Jul. 7, 2006.

(51) Int. Cl. C12Q 1/54; C12Q 1/00; G01N 33/00; G01N 33/48

(52) U.S. Cl. 435/7.92; 435/14; 435/4; 702/19

(57) ABSTRACT

A method for diagnosing a body weight condition or predisposition to a body weight condition in an animal by determining observed level(s) of at least one biomarker in a tissue or biofluid sample from the animal and comparing the observed level(s) to reference level(s) for the biomarker; wherein the observed level(s) relative to the reference level(s) are individually or collectively indicative of the body weight condition or predisposition.

### BACKGROUND OF THE INVENTION

1. Field of the Invention

The present invention relates to methods of diagnosing a body weight condition or predisposition in an animal. It also relates to a method for the calculating body condition score of an animal.

2. Description of the Prior Art

The prevalence of obesity has increased in both human and non-human populations. Obesity rates in humans are of epidemic proportions. Furthermore, studies show that 25% to 40% of all American household pets are overweight or obese, a trend that is leading to a steady rise in overweight-related pet illnesses and increased veterinary costs.

Being overweight can be a risk factor for development of a variety of disorders or diseases. Obesity, for example, has been linked to heart disease, degenerative joint disease, diabetes and cancer, among other conditions. Further, an overweight animal may experience considerable problems through reduced mobility and decreased overall quality of life. Prevention of an overweight condition can have a lifelong impact and knowledge of the risk factors for development of such a condition can lead to improved prevention and treatment programs that optimize overall health.

Various biomarkers, including for example plasma leptin, have been associated with food intake and body lat. Shiiya et al. (2002) J. Clin. Endocrinol. Metab. 87(1):240-244 reported that plasma ghrelin concentrations were lower in obese than in lean humans.

Despite awareness of the health implications of an overweight condition, treating such a condition remains a challenge due to, among other things, little understanding of the underlying physiological mechanisms or chances that occur in physiological systems that maintain such a condition. Measurement of body weight by traditional techniques is helpful, but the information thus gained is crude and may provide little insight into underlying physiological or biochemical processes associated with a body weight condition such as obesity. Furthermore, such traditional techniques have limited value in detecting or diagnosing a predisposition to obesity or other body weight condition in an animal having normal body weight. For example, the “body condition score” (BCS) of an animal has routinely been used as a means to classify an animal's body composition. Determination of an animal's BCS is based upon a visual and tactile analysis of an animals body size and shape by an animal health care professional. For example, according to this method, a BCS of “1” indicates an emaciated animal, “2” indicates a thin animal, a BCS of “3” indicates an optimal body condition for the animal, “4” indicates a fat animal and a BCS of 5 indicates an obese animal. Determination of an animal's CS is familiar to one of skill in the art; several methods are known to skilled artisans, e.g., methods disclosed in U.S. Pat. No. 6,691,639 and in the reference entitled “Small Animal Clinical Nutrition” 4^{th} Edition, in Chapter 13 (ISBN 0-945837-05-4). Although BCS determination is widely used, the method is less than ideal as it is a fairly subjective analysis with the not uncommon result that different individuals may determine an entirely different BCS for the same animal. Thus, there remains a need for effective methods for diagnosing a body weight condition or predisposition in an animal as well as for accurately determining the body condition score of such animal.

We have now surprisingly discovered and report herein a method to quantitate the body condition score of an animal based on biomarker data obtained from the animal. Such biomarker data may also be used to diagnose an animal's body weight condition and/or predisposition as well as to diagnose an obesity-related health disorder or predisposition thereto in an animal.

### SUMMARY OF THE INVENTION

The invention provides a method for diagnosing a body weight condition or predisposition thereto in an animal. The method comprises determining observed level(s) of at least one biomarker in a tissue or biofluid sample from the animal and comparing the observed level(s) to reference level(s) for the biomarker, wherein the observed level(s) relative to the reference level(s) are individually or collectively indicative of the body weight condition or predisposition.

The invention further provides a method to quantitate the body condition score for an animal comprising (a) analyzing the body weight and serum levels of at least one biomarker in said animal; and (b) applying said data obtained from step (a) to any of Algorithm I-IV of the invention described herein.

There is further provided a method for selecting a regimen for an animal. The method comprises (a) diagnosing a body weight condition or predisposition thereto by determining observed level(s) of at least one biomarker in a tissue or biofluid sample from the animal, and comparing the observed level(s) to reference level(s) for the biomarker, wherein the observed level(s) relative to the reference level(s) are individually or collectively indicative of the body weight condition or predisposition; and (b) identifying a regimen appropriate to the body weight condition or predisposition diagnosed.

There is still further provided a method for detecting onset of a body weight condition or predisposition in an animal. The method comprises monitoring at least one biomarker in the animal over a period by determining, at each of a plurality of time points during the period, observed level(s) of the biomarker in a tissue or biofluid sample from the animal, and comparing the observed level(s) to reference level(s) for the biomarker; wherein onset is detected if at any time point, the observed level(s) relative to the reference level(s) are individually or collectively indicative of the body weight condition or predisposition.

There is still further provided a method for assessing the efficacy of a regimen for managing a body weight condition or predisposition in an animal. The method comprises monitoring at least one biomarker in the animal over a period during which the regimen is administered, by determining, at each of a plurality of time points during the period, observed level(s) of the biomarker in a tissue or biofluid sample from the animal, and comparing the observed level(s) to reference level(s) for the biomarker; wherein the observed level(s) relative to the reference level(s) are individually or collectively indicative of the efficacy of the regimen in managing the body weight condition or predisposition.

There is still further provided a kit comprising:
(a) one or more reagents for detecting observed level(s) of at least one biomarker in a tissue or biofluid sample from an animal; and
(b) one or more user-accessible media carrying information that comprises (i) reference level(s) of the biomarker; and (ii) an algorithm that compares the observed level(s) to the reference level(s);
   wherein the observed level(s) relative to the reference level(s) are individually or collectively indicative of a body weight condition or predisposition in the animal.

There is still further provided a method for diagnosing an obesity-related health condition or a predisposition thereto in an animal comprising determining observed level(s) of at least one biomarker in a tissue or biofluid sample from the animal and comparing the observed level(s) to reference level(s) for the biomarker: wherein the observed level(s) relative to the reference level(s) are individually or collectively indicative of the body weight condition or predisposition. In particular, the method is useful for diagnosing osteoarthritis wherein the biomarker is alkaline phosphatase, osteocalcin, amino terminal crosslink telopeptide, Type II cartilage synthesis, cartilage oligomeric matrix protein, or carboxy terminal crosslink telopeptide. Preferably, the biomarker is alkaline phosphatase or Type II cartilage synthesis.

Additional objects, features, and advantages of the invention will be apparent to those skilled in the art.

### DETAILED DESCRIPTION OF THE INVENTION

It has been found in accordance with the invention that levels of certain biomarkers in a tissue or biofluid sample from an animal can be surprisingly effective for calculating the body condition score of an animal. In addition, biomarker data may also be used in a method for the diagnosis of a body weight condition in the animal. Levels of such biomarkers can fluctuate from a preprandial to a postprandial state. However, individual animals with different body weight conditions. e.g., lean and obese animals, show differences in the form and/or degree of such fluctuation, as well as in absolute levels of the biomarkers when in a fasted state. Profiles of one or more biomarkers, therefore, can be indicative of a body weight condition. Further, such profiles are indicative of a predisposition to a body weight condition, even where that condition is not yet expressed, and also may be used to diagnose an obesity-related health disorder in an animal or a predisposition thereto. Thus, such profiles are useful in managing an animal's body weight and health consequences that may be associated with a body weight condition existing in the animal or to which the animal is predisposed.

Biomarkers of interest herein are those for which an observed level relative to a reference level is indicative of a body weight condition or predisposition. According to some embodiments, a reference level can be established from samples obtained from healthy animals of normal body weight, or can be a published value. Typically, reference levels are established for animals of the same species and, if possible breed or breed type. Further, it is generally preferable that reference levels are established for animals of similar age group to the animal. Determination of such reference levels, including determining the “normal” body weight of an animal, would be familiar to one of skill in the art. In this case, an observed level substantially different from (e.g., higher or lower than) the reference level can be indicative of a body weight condition or predisposition. Such a difference can be, but is not necessarily, statistically significant.

Alternatively, a reference level can be established for animals known to have a particular body weight condition or predisposition; an observed level similar to the reference level can in this case be indicative of the condition or predisposition.

The level of a biomarker can provide information about underlying genetic, biochemical or physiological factors, mechanisms or pathways associated with a particular existing body weight condition (e.g., normal weight, overweight, obese), but is not necessarily informative in this way. In some cases, a statistical correlation between a level of a biomarker and an observed body weight condition or predisposition can suffice for practice of the invention. However, where the biomarker provides information of genetic, biochemical or physiological relevance, advantages over traditional methods relying solely on physical measurements related to body weight can be especially great.

The animal can be human or non-human. In various embodiments, the animal is a vertebrate, for example a fish, a bird, a reptile or a mammal. Illustratively among mammals, the animal can be a member of the order Carnivora, including without limitation canine and feline species. In one embodiment, the animal is a cow, horse, pig or other form of domestic livestock for which determination of body condition score and/or body weight condition is important.

In an embodiment, the animal is a companion animal. A “companion animal” herein is an individual animal of any species kept by a human caregiver as a pet, or any individual animal of a variety of species that have been widely domesticated as pets, including dogs (Canis familiaris) and cats (Felis domesticus), whether or not the individual animal is kept solely or partly for companionship. Thus “companion animals” herein include working dogs, farm eats kept for rodent control. etc. as well as pet dogs and cats. In some embodiments, the animal is a canine. In other embodiments, the animal is a feline.

It is also contemplated herein that the methods of the present invention may be applied to humans, for example, for the quantitation of the “body condition score” of a human, more usually referred to as “body mass index” or BMI. As BMI typically refers to an animal's weight (in kilograms) divided by its height (in meters) squared conversion of the algorithms disclosed herein for calculation of BMI may be required. This may easily be achieved by one of skill in the art.

A “body weight condition” diagnosed according to the invention based on biomarker analysis against a reference sample can be, for example, a determination that an animal is underweight of normal weight, is overweight or obese. Body weight is generally not simply a matter of weight alone, but is usually associated with quantity or percentage of body fat. See for example Burkholder & Toll (2000) in Hand et al. (eds.). Small Animal Clinical Nutrition. 4th Edition, Chapter 13, pp 402-430.

A “body weight predisposition” as used herein refers to an animal's proneness (i.e., propensity) for gaining, losing, or maintaining body weight and/or undergoing concomitant changes in health or other physiological conditions. Thus examples of body weight predispositions include a propensity, or lack thereof, to gain weight and a predisposition to obesity.

The “body condition score” (BCS) of an animal as used herein refers to a means to classify an animal's body composition. Determination of an animal's BCS has traditionally been based upon a visual and tactile analysis of an animal's body size and shape by an animal healthcare professional. According to this traditional method, a BCS of “1” indicates an emaciated animal. “2” indicates a thin animal, a BCS of “3” indicates an optimal body condition for the animal, “4” indicates a fat animal and a BCS of 5 indicates an obese animal. Conventional determination of an animal's BCS is familiar to one of skill in the art several methods are known to skilled artisans, e.g., methods disclosed in U.S. Pat. No. 6,691,639 and in the reference entitled “Small Animal Clinical Nutrition”, 4^{th} Edition, in Chapter 13 (ISBN 0-945837-05-4).

According to one embodiment, a body weight predisposition is diagnosed by a method of the invention while the animal is young, for example, in the case of a canine or feline, up to about one year of age.

An overweight or obese condition can be an associative cause or exacerbating factor for a number of diseases and disorders. Such obesity-related diseases and health disorders include, for example, metabolic alterations, endocrinopathies, functional alterations, degenerative joint and orthopedic diseases, cardiovascular diseases, cancers, sleep disorders, reproductive disorders, and combinations thereof. An overweight condition also can cause considerable problems through reduced mobility or decreased quality of life. In one embodiment, therefore, a body, weight condition or predisposition diagnosed by practice of the invention is one that increases the animal's risk for an obesity-related health disorder.

Such overweight or obesity-related heath disorders illustratively include hyperlipidemia, dyslipidemia, insulin resistance, glucose intolerance, hepatic lipidosis, anesthetic complications, hyperadrenocorticism, hypothyroidism, diabetes mellitus, insulinoma, pituitary chromophobe adenoma, hypopituitarism, hypothalamic lesions, joint stress, musculoskeletal pain, dyspnea, hypertension, dystocia, exercise intolerance, heat intolerance, decreased immune function, degenerative joint and orthopedic diseases (e.g., osteoarthritis), cardiovascular diseases, hypertension, respiratory distress, altered kidney function, pancreatitis, transitional cell carcinomas, fatigue, sleep disorders, reproductive disorders, and combinations thereof.

The term “biomarker” means a substance that can be quantitatively identified in a tissue or biofluid sample and that provides a correlation to a particular phenotype or physiological condition. Illustratively, a biomarker can be a cytokine, e.g., an inflammatory cytokine; a peptide or protein, e.g., peptide YY, neuropeptide Y, glucagon-like peptide I (GLP-I), alkaline phosphatase, ghrelin; a nucleic acid, e.g., an mRNA transcript corresponding to a peptide or protein biomarker, a biochemical metabolite, e.g., glucose; a neurotransmitter; an agonist; an antagonist; or other biomarkers such as thyroxine, thyroid stimulating hormone, insulin like growth factor-1, leptin, angiotensin I and II, c-reactive protein, high density lipoprotein 1 and 2, low density lipoprotein, very low density lipoprotein, chylomicron, testosterone, estradiol, cortisol, osteocalcin, amino terminal crosslink protein, type 11 cartilage synthesis or cartilage oligomeric matrix protein. In various embodiments, level(s) of at least one of the following biomarkers are determined: glucose, GLP-1, ghrelin, leptin, adiponectin, resistin, resistin-like molecules, c-reactive protein, thyroid stimulating hormone and insulin. Particularly useful biomarkers include glucose, GLP-1, c-reactive protein thyroid stimulating hormone, and ghrelin.

The term “obesity biomarker” herein refers to a substance that can be quantitatively identified in a tissue or biofluid sample and that can provide a correlation to obesity. Examples of obesity biomarkers include, but are not limited to, cholesterol, triglycerides, glucagon like protein-1, insulin like growth factor-1, ghrelin leptin, GLP-1, angiotensin I and II, high density lipoprotein-1, high density lipoprotein-2, low density lipoprotein and very low density lipoprotein.

The term “arthritis biomarker” as used herein refers to a substance that can be quantitatively identified in a tissue or biofluid sample and that call provide a correlation to conditions characterized by damage to the joints of the body, e.g., arthritis or osteoarthritis. Examples of arthritis biomarkers include, but are not limited to, osteocalcin, amino terminal crosslink telopeptide, alkaline phosphatase, carboxy terminal crosslink telopeptide, Type II cartilage synthesis and cartilage oligomeric matrix protein.

The term “thyroid biomarker” as used herein refers to a substance that can be quantitatively identified in a tissue or biofluid sample and that can provide a correlation to a thyroid disease or disorder. Examples of thyroid biomarkers include, but are not limited to thyroid stimulating hormone and thyroxine.

Diagnosis of a body weight condition or predisposition by the method of the invention can involve determination of more than one biomarker. In some cases, a single biomarker can be indicative of the body weight condition or predisposition; in other cases, a biomarker profile comprising levels of two or more biomarkers, is collectively indicative of the condition or predisposition. In other cases, a profile comprising levels of at least one biomarker and other blood chemicals such as sodium, potassium, chloride, phosphorus, bilirubin, creatinine, or serum urea nitrogen, is collectively indicative of the condition or predisposition.

Any tissue or biofluid sample can be a source of biomarkers of interest. However, in most cases biofluid samples that can be obtained with minimal invasion are preferred. Biofluids illustratively include whole blood, blood serum, blood plasma, cerebrospinal fluid, crevicular fluid, urine, lymph fluid, intramuscular fluid, nasal secretion and saliva.

A level of a biomarker can be determined using assays known in the art. An assay can, but need not, be a commercially available assay. Typically, an assay is chosen based on the type of biomarker and the type of sample. For example, a commercially available monoclonal-based immunoassay utilizing monoclonal antibodies reactive to one or more epitopes on polypeptides or a competitive binding assay can be used for determining a blood serum level of a protein biomarker such as, for example, GLP-1 or ghrelin; and an assay based on a ferricyanide, hexokinase, or glucose oxidase procedure can be used for determining a blood serum level of glucose.

In some embodiments, observed and/or reference levels are determined using one or more assays independently selected from the group consisting of enzyme immunoassays (EIA), enzyme-linked immunosorbent assays (ELISA), immunofluorescent assays (IFA), radioimmunoassays (RIA), western blot assays, biochemical assays, enzymatic assays, and colorimetric assays. A variety of labels and conjugation techniques are known by those skilled in the art and can be used in the various biochemical, nucleic acid and amino acid assays.

A tissue or biofluid sample can be collected, for example, at a point of care facility, i.e., a place where an animal can be seen by a health care practitioner (e.g. medical doctor, veterinarian, medical assistant, physician's assistant, nurse, etc.) for evaluation and diagnosis. Non-limiting examples of a point of care facility include a hospital, office of a physician or veterinarian, and veterinary clinic. Alternatively, a sample can be collected at the animal's home, farm, stable or barracks where the animal is kept.

Analysis of the sample for the one or more biomarkers of interest can be done at the place, e.g. point of care facility, where the sample is taken. A kit as described herein can be used in such analysis. Alternatively, the sample can be sent to a secondary facility. The term “secondary facility” means a laboratory such as a commercial testing laboratory where clinical samples are evaluated and can be off-site (i.e. at a different location) from a point of care facility.

In some embodiments, comparing the observed level(s) to reference level(s) of the one or more biomarkers is performed at a point of care facility or a secondary facility.

Where a sample is taken at a single time point, this can be at any stage of the animal's feeding cycle, for example immediately before a meal (preprandial) or at a suitable interval after a meal (postprandial). However, it is generally preferred that when diagnosis is to be based on a single sample, that such sample be taken when the animal is in a fasting state, for example at a preprandial time point.

Optionally, samples are taken at a plurality of time points during the feeding cycle. In this case, at least one (typically just one) preprandial sample and at least one (typically more than one) postprandial sample can be taken. Suitable time points are illustratively 0 (preprandial), 10, 30, 60, 120 and 360 minutes postprandial.

Biomarker levels in a sample, for example a serum sample, can be unadjusted, or adjusted for body weight of the animal. Unadjusted levels can be expressed in weight/volume concentration units such as mg/L, μg/L or ng/L, or molar concentration units such as μmol/L, nmol/L or pmol/L. Adjusted levels can be expressed in similar units, but with body weight (BW) as a divisor, e.g. mg/L/kg BW, pmol/L/kg BW, etc.

In one embodiment, the animal is canine and the biomarker comprises glucose in serum. According to this embodiment, an observed body weight-adjusted serum glucose level in a fasted animal at least about 10% lower than the body weight-adjusted reference level for a canine of normal weight is indicative of a predisposition of the animal to gain weight.

In another embodiment, the animal is canine and the biomarker comprises GLP-1 in serum. According to this embodiment, an observed body weight-adjusted serum GLP-1 level in a fasted animal at least about 20% lower than the body weight-adjusted reference level for an animal of normal weight is indicative of a predisposition of the animal to gain weight.

In yet another embodiment, the animal is canine and the biomarker comprises ghrelin in serum. According to this embodiment, an observed body weight-adjusted serum ghrelin level in a fasted animal at least about 20% lower than the body weight-adjusted reference level for a canine of normal weight is indicative of a predisposition of the animal to gain weight.

In yet another embodiment, the invention relates to a method to quantitate the body condition score of an animal comprising (a) analyzing the body weight and serum levels of glucose, sodium, chloride, c-reactive protein and thyroid stimulating hormone of said animal; and (b) applying the data obtained from step (a) to the following algorithm

Body condition score=3.62352+(0.17443×body weight in kg)+(0.01621×glucose in mg/dL)+(0.06496×sodium in mmol/L)−(0.12439×chloride in mmol/L)−(0.05575×c-reactive protein in ng/mL)+(1.72392×thyroid stimulating hormone in ng/mL).

In a particular embodiment, the animal is a canine.

In yet another embodiment, the invention relates to a method to quantitate the body condition score of an animal comprising (a) analyzing the body weight and serum levels of urea nitrogen, sodium and chloride in said animal; and (b) applying the data obtained from step (a) to the following algorithm

Body condition score = 3.64120 + (0.18614 × body weight in kg) −
(0.05289 × serum urea nitrogen in mg/dL) + (0.08935 ×
sodium in mmol/L) − (0.14088 × chloride in mmol/L).

In a particular embodiment, the animal is a canine.

In yet another embodiment, the invention relates to a method to quantitate a body condition score in an animal comprising (a) analyzing the body weight and serum levels of sodium, potassium, chloride, phosphorus, bilirubin and ghrelin in said animal, and (b) applying the data obtained from step (a) to the following algorithm:

Body condition score = −3.20078 + (0.4259 × body weight in kg) −
(0.05508 × sodium in mmol/L) + (0.69884 × potassium in mmol/L) +
(0.09472 × chloride in mmol/L) − (0.15372 × phosphorus in mg/dL) +
(1.31580 × total bilirubin in mg/dL) − (0.35136 × ghrelin in ng/mL).

In a particular embodiment, the animal is a feline.

In yet another embodiment, the invention relates to a method to quantitate a body condition score in an animal comprising (a) analyzing the body weight and serum levels of blood urea nitrogen:creatinine ratio, potassium, chloride, phosphorus and total bilirubin in said animal; and (b) applying the data obtained from step (a) to the following algorithm:

Body condition score = −7.34191 + (0.48335 × body weight in kg) +
(0.03578 × blood urea nitrogen:creatinine) + (0.58860 ×
potassium in mmol/L) + (0.04683 × chloride in mmol/L) − (0.16894 ×
phosphorus in mg/dL) + (0.86613 × total bilirubin in mg/dL).

In a particular embodiment, the animal is a feline.

Upon diagnosis of a body condition score, body weight condition or predisposition as described above, a regimen appropriate to the condition or predisposition can be selected. The regimen can be selected by the animal or the animal's caregiver based on information communicated by any suitable communication means, or can be prescribed by a health care professional. The regimen can comprise one or more of diet, exercise, and medication, in some embodiments, a regimen comprises a composition for consumption by the animal. Illustratively such a composition can be a nutritional composition, such as a food composition, a supplement, a treat or a toy, it being noted that some, but not all, supplements, treats and toys are them selves food compositions. Food compositions can be, for example, ingested by an animal or administered to an animal by feeding. Where the animal is a companion animal, a food composition useful in the method of the invention is typically one that is nutritionally adapted for feeding to such an animal (referred to herein as a “pet food”) and is appropriate for the body weight condition or predisposition diagnosed. Pet foods can be more particularly adapted to the special nutritional needs of canines or felines, or to certain subpopulations thereof such as large-breed dogs, puppies or kittens, young dogs or cats, adult dogs or cats, senior dogs or cats, and Geriatric dogs or cats.

A food composition forming part of a regimen can be one providing a substantially nutritionally complete diet for the animal. A “nutritionally complete diet” is a diet that includes sufficient nutrients for maintenance of normal health of a healthy animal on the diet.

Alternatively, the composition can be a supplement, i.e., a composition used with another food composition to improve the nutritive balance or performance of the diet as a whole. Such supplements include food compositions that are fed undiluted as a supplement to other foods, offered free choice with other parts of an animal's ration that are separately available to the animal, or diluted and mixed with an animal's regular food to produce a substantially nutritionally complete diet. Supplements can alternatively be in a form other than a food composition, for example in a pharmaceutical-like dosage form including, for example, powders, liquids, syrups, pills, etc.

The composition can be a treat. Treats include, for example, compositions given to an animal as a reward or to entice the animal to eat during a non meal time. Treats for dogs that are food compositions having at least some nutritional value include, for example, dog biscuits. Treats can alternatively be substantially non-nutritional. A composition forming part of a regimen can itself form a treat, be coated onto an existing treat, or both.

The composition can be a toy adapted for oral use by an animal. Toys include, for example, chewable toys, such as artificial bones for dogs. A composition useful herein can for a coating on the surface of a toy or on the surface of a component of a toy, be incorporated partially or fully throughout the toy, or both. A wide range of suitable toys are currently marketed, including partially consumable toys (e.g., toys comprising plastic components) and fully consumable toys (e.g., rawhides and various artificial bones). Toys are available for human and non-human use, particularly for companion, farm, and zoo animal use, and more particularly or dog, cat, or bird use.

In other embodiments, a regimen comprises a form of exercise. Exercise can take any form suitable for the animal and appropriate for the body weight condition or predisposition diagnosed. Illustratively, exercise can include without limitation walking jogging or running.

The regimen can be continued at a frequency or for a period of time as is necessary or appropriate for the body weight condition or predisposition. Illustratively, a regimen can continue for at least about 1 month, at least about 2 months, at least about 6 months, at least about 1 year, or for some other period of time as may be determined necessary or appropriate, for example by a veterinarian or other health care professional.

The invention also provides a method for detecting onset of a body weight condition or predisposition in an animal. According to this method, at least one biomarker in the animal is monitored over a period, and onset is detected it, at any time point during that period, the observed level(s) relative to the reference level(s) of the biomarker are individually or collectively indicative of the body weight condition or predisposition.

Such a method optionally further comprises monitoring the animal's body weight during at least part of the period. Any appropriate technique for determining body weight can be used, including without limitation weighing, assessment of relative body weight (RBW), assessment of body condition score (BCS), morphometry, and combinations thereof. Additional useful information relating to body weight can optionally be obtained by techniques such as magnetic resonance imaging (MRI), computerized tomography (CT), neutron activation, hydrodensitometry, total body water by isotope dilution, total body potassium, ultrasound, bioelectrical impedance, radiograph, sonograph, dual energy x-ray absorptiometry (DEXA), or combinations thereof.

Monitoring of the biomarker, and optionally of body weight and/or other related parameters, can be performed at any convenient interval, for example at about hourly, twice daily, daily, twice weekly, weekly, monthly, bimonthly, twice yearly or yearly intervals.

Monitoring of the biomarker can also provide a useful method for assessing the efficacy of a regimen for managing a body weight condition or predisposition in an animal. According to this method, the biomarker, and optionally body weight and/or other related parameters, are monitored over a period during which the regimen is administered. The observed level(s) relative to the reference level(s) of the biomarker can be individually or collectively indicative of the efficacy of the regimen in managing the body weight condition or predisposition.

In another embodiment of the invention, a kit is provided, suitable for use according to any of the methods described herein. Such a kit comprises one or more reagents for detecting observed level(s) of at least one biomarker in a tissue or biofluid sample from an animal; and one or more user-accessible media carrying information that comprises (i) reference level(s) of the biomarker; and (ii) an algorithm that compares the observed level(s) to the reference level(s). As in previous embodiments, the observed level(s) relative to the reference level(s) are individually or collectively indicative of a body weight condition or predisposition in the animal.

“User-accessible” media herein include all media, such as paper, disk, memory chip, card, computer or network, on which instructions, information, an algorithm and or data can be retrievably contained or stored. The algorithm is typically a software algorithm. One example of a “user-accessible media” is the SAS/STAT® Software, which uses the regression procedure to determine the algorithm for predicting body condition score. Examples of such an algorithm are Algorithm I-IV provided in the examples described below, e.g.,

| | |
|---|---|
| Algorithm I: | Body condition score = 3.62352 + (0.17443 × |
| | body weight in kg) + (0.01621 × glucose in mg/dL) + |
| | (0.06496 × sodium in mmol/L) − (0.12439 × chloride in |
| | mmol/L) − (0.05575 × c-reactive protein in ng/mL) + |
| | (1.72392 × thyroid stimulating hormone in ng/mL) |
| Algorithm II: | Body condition score = 3.64120 + (0.18614 × |
| | body weight in kg) − (0.05289 × serum urea nitrogen |
| | in mg/dL) + (0.08935 × sodium in mmol/L) − |
| | (0.14088 × chloride in mmol/L) |
| Algorithm III: | Body condition score = −3.20078 + (0.4259 × body |
| | weight in kg) − (0.05508 × sodium in mmol/L) + |
| | (0.69884 × potassium in mmol/L) + (0.09472 × chloride |
| | in mmol/L) − (0.15372 × phosphorus in mg/dL) + |
| | (1.31580 × total bilirubin in mg/dL) − (0.35136 × |
| | ghrelin in ng/mL) |
| Algorithm IV: | Body condition score = −7.34191 + (0.48335 × body |
| | weight in kg) + (0.03578 × blood urea |
| | nitrogen:creatinine) + (0.58860 × potassium in |
| | mmol/L) + (0.04683 × chloride in mmol/L) − |
| | (0.16894 × phosphorus in mg/dL) + (0.86613 × |
| | total bilirubin in mg/dL). |

The kit is optionally self-contained so as not to require laboratory equipment. Optionally, the kit further comprises a tissue or biofluid sample collection device. The kit can employ one or more of a variety of assays for determining a level of a biomarker, including the assays listed above, Standards and standard additions can be included and used for calibration in quantifying the level of a biomarker in a sample, using well known techniques.

In some embodiments, the one or more reagents of the kit comprise a reporter moiety or label. The reporter moiety or label can illustratively comprise biotin, a chromogenic agent, a luminescent or chemiluminescent, a cofactor, an enzyme, a fluorescent agent, an inhibitor, a metal or magnetic particle, a radionuclide, a substrate or a combination thereof, and can be detected using methods known in the art. Illustratively, such methods include without limitation spectroscopic methods used to detect dyes (including, for example, colorimetric detection of products of enzyme reactions), luminescent groups and fluorescent groups; detection of enzyme reporter groups by addition of a substrate, followed by spectroscopic, spectrophotometric or other analysis of reaction products; scintillation counting or autoradiographic methods for radioactive groups; and Rarnan scattering techniques for metal nanoparticles (e.g., gold nanoparticles).

The one or more reagents of a kit can comprise at least one antibody, for example a polyclonal or monoclonal antibody. The antibody can be immobilized on a solid support. For example, an ELISA can be utilized to determine a level of a biomarker in a sample. The ELISA can involve coupling an antibody onto a solid support such as a polymer. A sample comprising a biomarker can be introduced and the biomarker allowed to interact with the antibody, whereupon a signal (e.g., chromogenic signal) generating process can be performed to create an optically detectable signal.

In one embodiment, the kit comprises a first antibody that specifically binds to the biomarker in the sample, and a second antibody that specifically binds to the resulting complex of the first antibody and the biomarker. The second antibody can be immobilized to a solid support. For example, upon binding of the second antibody to the first antibody/biomarker complex, the second antibody can trigger a reaction and, for example, result in a detectable color change.

A variety of labels and conjugation techniques are known by those skilled in the arts. Techniques for producing labeled hybridization or PCR probes for detection and quantification of nucleic acid sequences include oligo-labeling, nick translation, end labeling and PCR amplification using a labeled nucleotide. Alternatively, the coding sequence of a biomarker, or any portion thereof, may be cloned into a vector for production of an mRNA probe. Such vectors are known in the art, are commercially available, and can be used to synthesize RNA probes in vitro by addition of an appropriate RNA polymerase such as T7, T3 or SP6, and labeled nucleotides.

The kit optionally further comprises means for communicating information comprising one or more of (a) a diagnosis of a body weight condition or predisposition as indicated by the observed level(s) relative to the reference level(s) of the biomarker; and (b) a suggested or prescribed regimen appropriate to the diagnosis.

The communicating means can be attached to or enclosed in a package containing other elements of the kit. Any suitable form of communicating means can be employed, for example a document such as a label, brochure, advertisement or package insert, a computer readable digital or optical medium such as a diskette or CD, an audio presentation, for example on an audiotape or CD, or a visual presentation, for example on a videotape or DVD. The communicating means can refer to further information located elsewhere, such as on a website.

Such a communicating means, comprising for example a document such as a label, brochure, advertisement or package insert, a computer-readable digital or optical medium such as a diskette or CD, an audio presentation, for example on an audiotape or CD, a visual presentation, for example on a videotape or DVD, and/or one or more pages on a website, is itself a still further embodiment of the invention.

The invention is not limited to the particular methodology, protocols, and reagents described herein because they may vary. Further, the terminology used herein is for the purpose of describing particular embodiments only and is not intended to limit the scope of the present invention. As used herein and in the appended claims, the singular forms “a”, “an”, and “the” include plural reference unless the context clearly dictates otherwise. Similarly, the words “comprise”, “comprises”, and “comprising” are to be interpreted inclusively rather than exclusively.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the preferred methods, devices, and materials are described herein.

All patents, patent applications, and publications mentioned herein are incorporated herein by reference to the extent allowed by law for the purpose of describing and disclosing the compounds, processes, techniques, procedures, technology, articles, and other compositions and methods disclosed therein that might be used with the present invention. However, nothing herein is to be construed as an admission that the invention is not entitled to antedate such disclosure by virtue of prior invention.

### EXAMPLES

The invention can be further illustrated by the following examples of preferred embodiments thereof, although it will be understood that these examples are included merely for purposes of illustration and are not intended to limit the scope of the invention unless otherwise specifically indicated.

### Example 1

### Biomarkers in Lean and Obese Animals

This example illustrates that levels of certain biomarkers, relative to reference levels, can be indicative of a body weight condition or predisposition in an animal.

Twenty dogs (ten lean and ten obese) were used in a four day study to determine differences in serum metabolites between lean-prone and obese-prone dogs. Placement in the lean or obese group was determined by the following characteristics: (a) propensity to gain weight (obese-prone) or to maintain weight (lean-prone) when fed ad libitum; (b) numerical body condition score ranging from 1 to 5 (lean-prone dogs had an average body condition score of about 3, whereas obese-prone dogs had an average body condition score of about 4.1) and (c) past participation in weight loss studies (obese dogs had previous participation, whereas lean dogs had not).

Average body weight for lean-prone dogs was 12.06 kg and for obese-prone dogs 16.59 kg.

The dogs were fed, once daily for four days, a maintenance food formulated to meet or exceed nutritional requirements for maintenance of body weight (BW). On the fourth day, blood serum samples were taken prior to feeding (preprandial, time 0), and 10, 60, 120, and 360 minutes after feeding (postprandial). The samples were analyzed for insulin, triglycerides, glucose. GLP-1, and ghrelin concentrations using standard procedures found, for example, in laboratory manuals such as Sambrook et al. (2001) Molecular Cloning: A Laboratory Manual, 3rd ed. Cold Spring Harbor Laboratory Press, Cold Spring Harbor, N.Y.; Spector et al. (1998) Cells: A Laboratory Manual, Cold Spring Harbor Laboratory Press, Cold Spring Harbor, N.Y.; and Hampton et al. (1990) Serological Methods: A Laboratory Manual, APS Press, St Paul, Minn. Serum concentrations of the biomarkers were adjusted for BW. Results are shown in Tables 1 through 5.

Postprandial serum insulin concentrations did not differ substantially between lean-prone and obese-prone dogs (Table 1).

TABLE 1
Serum Insulin Levels

| | Insulin concentration | | BW-adjusted insulin | |
|---|---|---|---|---|
| Time | (pmol/l) | | (pmol/l/kg BW) | |
| (min) | Lean-prone | Obese-prone | Lean-prone | Obese-prone |
| 0 | 50.8 | 76.3 | 4.5 | 4.7 |
| 10 | 87.4 | 280.3 | 10.3 | 17.9 |
| 30 | 65.2 | 335.1 | 5.6 | 21.5 |
| 60 | 138.4 | 200.9 | 12.1 | 12.8 |
| 120 | 101.2 | 235.0 | 8.2 | 14.3 |
| 360 | 41.3 | 126.9 | 7.0 | 7.1 |

Also, serum triglyceride concentrations did not differ substantially between lean-prone and obese-prone dogs (Table 2).

TABLE 2
Serum Triglyceride Levels

| | Triglyceride concentration | | BW-adjusted triglycerides | |
|---|---|---|---|---|
| Time | (mg/dI) | | (mg/dl/kg BW) | |
| (min) | Lean-prone | Obese-prone | Lean-prone | Obese-prone |
| 0 | 62.9 | 75.9 | 5.6 | 4.6 |
| 10 | 66.9 | 87.2 | 5.9 | 5.3 |
| 30 | 79.6 | 126.1 | 7.0 | 7.6 |
| 60 | 131.8 | 219.5 | 11.7 | 13.3 |
| 120 | 175.9 | 281.8 | 15.9 | 17.1 |
| 360 | 90.5 | 211.9 | 7.6 | 12.8 |

Serum concentrations of glucose (Table 3), GLP-1 (Table 4), and ghrelin (Table 5) levels differed substantially between lean-prone and obese-prone dogs at most or all of the six sampling times.

TABLE 3
Serum Glucose Levels

| | Glucose concentration | | BW-adjusted glucose | |
|---|---|---|---|---|
| | (mg/dl) | | (mg/dl/kg BW) | |
| Time (min) | Lean-prone | Obese-prone | Lean-prone | Obese-prone |
| 0 | 86.2 | 91.3 | 7.4 | 5.6 |
| 10 | 91.8 | 94.3 | 7.8 | 5.4 |
| 30 | 92.5 | 94.3 | 7.9 | 5.8 |
| 60 | 93.8 | 90.4 | 8.0 | 5.5 |
| 120 | 89.9 | 88.3 | 7.6 | 5.4 |
| 360 | 85.8 | 88.8 | 7.3 | 5.4 |

TABLE 4
Serum GLP-1 Levels

| | GLP-1 concentration | | BW-adjusted GLP-1 | |
|---|---|---|---|---|
| | (pmol/l) | | (pmol/l/kg BW) | |
| Time (min) | Lean-prone | Obese-prone | Lean-prone | Obese-prone |
| 0 | 15.8 | 7.9 | 1.34 | 0.50 |
| 10 | 17.0 | 13.5 | 2.07 | 0.85 |
| 30 | 25.5 | 15.1 | 2.19 | 0.94 |
| 60 | 25.5 | 15.5 | 2.15 | 0.97 |
| 120 | 27.8 | 18.0 | 2.33 | 1.13 |
| 360 | 23.0 | 13.5 | 1.86 | 0.85 |

TABLE 5
Serum Ghrelin Levels

| | ghrelin concentration | | BW-adjusted ghrelin | |
|---|---|---|---|---|
| | (ng/rnl) | | (ng/ml/kg BW) | |
| Time (min) | Lean-prone | Obese-prone | Lean-prone | Obese-prone |
| 0 | 4.10 | 3.10 | 0.347 | 0.189 |
| 10 | 3.83 | 2.90 | 0.327 | 0.176 |
| 30 | 3.60 | 2.76 | 0.310 | 0.170 |
| 60 | 3.42 | 2.51 | 0.289 | 0.152 |
| 120 | 3.06 | 2.68 | 0.257 | 0.162 |
| 360 | 2.77 | 3.20 | 0.320 | 0.193 |

Referring to the Tables, the data shows the utility of biomarkers, in particular serum glucose, GLP-1 and ghrelin levels, to differentiate animals having lean and obese predisposition.

### Example 2

### Prediction of Body Weight Predisposition in Dogs

Thirty lean and thirty overweight dogs were identified for this study. Dogs with a body condition score (BCS) of 4 or 5 were classified as overweight for purposes of this study (on a scale of 1 through 5 where 1 equals thin and 5 equals obese/overweight). Dogs with a BCS less than 3 were classified as lean. The dogs were cared for in accordance with Hill's Institutional Animal Care and Use Committee protocols. Fifty percent of the dogs were female (15 lean and 15 overweight) and fifty percent were male (15 lean and 15 overweight) in order to determine it gender played a role in any marker differences. All animals were spayed or neutered because these groups of animals are more prone to obesity. Animals were weighed, given a body condition score and a blood sample was drawn. Serum was harvested and stored at −20° C. in 1 mL aliquots.

Serum was analyzed for chemistry screens, obesity markers, thyroid markers and arthritis markers. Chemistry screens were preformed at the Hill's Pet Nutrition Center (Topeka, Kans.). Insulin analysis was performed by Michigan State University (Lansing, Mich.). Thyroxine, thyroid stimulating hormone, glucagon like protein-1, insulin like growth factor-1, ghrelin, leptin, angiotensin I and II, c-reactive protein, high density lipoprotein 1 and 2, low density lipoprotein, very low density lipoprotein, chylomicron, testosterone, estradiol, cortisol, osteocalcin, amino terminal crosslink protein, type 2 cartilage synthesis and cartilage oligometric protein were performed by MD Biosciences, Inc. (St Paul, Minn.).

Data were analyzed using General Linear Models procedure of SAS (1989) to determine treatment means. The experimental unit was dog. Differences were considered significant when P<0.05 and trends were determined when P<0.10.

TABLE 2-1
Average Measurements for Lean and Overweight Dogs

| | | Average | | |
|---|---|---|---|---|
| | Average Lean | Overweight | Standard | Lean vs |
| Measurement | (n = 30) | (n = 30) | Error | Overweight* |
| Age, years | 8.25 | 6.70 | 0.65 | 0.10 |
| Body Condition Score | 2.53 | 4.70 | 0.08 | <0.01 |
| Body Weight, kg | 11.18 | 17.26 | 0.42 | <0.01 |
| Glucose, mg/dL | 84.10 | 93.20 | 2.00 | <0.01 |
| Insulin, pmol/L | 67.33 | 126.54 | 14.1 | <0.01 |
| Alanine Amino-transferase, U/L | 53.40 | 55.57 | 9.71 | NS |
| Alkaline Phosphatase, U/L | 115.28 | 191.30 | 25.17 | 0.04 |
| Cholesterol, mg/dL | 201.93 | 228.37 | 9.06 | 0.04 |
| Triglycerides, mg/dL | 91.87 | 242.77 | 56.53 | 0.06 |
| Total Bilirubin, mg/dL | 0.40 | 1.36 | 0.48 | NS |
| Total Protein, g/dL | 6.28 | 6.97 | 0.14 | <0.01 |
| Creatinine, mg/dL | 0.68 | 0.60 | 0.02 | 0.01 |
| Serum Urea Nitrogen, mg/dL | 12.45 | 9.50 | 0.48 | <0.01 |
| Albumin:Globulin | 1.20 | 1.18 | 0.06 | NS |
| Albumin, g/dL | 3.35 | 3.63 | 0.06 | <0.01 |
| Thyroxine, ug/dL | 1.71 | 2.02 | 0.11 | 0.05 |
| Thyroid Stimulating Hormone, ng/mL | 0.19 | 0.21 | 0.02 | NS |
| Calcium, mg/dL | 9.92 | 10.49 | 0.13 | <0.01 |
| Phosphorous, mg/dL | 4.19 | 4.65 | 0.15 | 0.04 |
| Chloride, mmol/L | 116.27 | 113.50 | 0.45 | <0.01 |
| Potassium, mmol/L | 4.52 | 4.43 | 0.06 | NS |
| Magnesium, mg/dL | 2.74 | 2.83 | 0.07 | NS |
| Sodium, mmol/L | 158.00 | 157.47 | 0.51 | NS |
| Sodium:Potassium | 35.17 | 35.73 | 0.46 | NS |
| Glucagon Like Protein-1, pM | 7.38 | 13.09 | 2.31 | 0.09 |
| Insulin Like Growth Factor-1, ng/mL | 102.0 | 183.6 | 17.4 | <0.01 |
| Ghrelin, ng/mL | 2.60 | 2.04 | 0.23 | 0.09 |
| Leptin, ng/mL | 0.96 | 5.14 | 0.49 | <0.01 |
| Angiotensin I, ng/mL | 0.61 | 0.66 | 0.05 | NS |
| Angiotensin II, ng/mL | 0.67 | 1.22 | 0.33 | NS |
| C-reactive Protein, ng/mL | 5.54 | 2.54 | 0.62 | <0.01 |
| Non-esterified fatty acids, mM | 0.75 | 0.80 | 0.07 | NS |
| High Density Lipoprotein-1, % of total | 15.4 | 11.5 | 1.6 | 0.10 |
| High Density Lipoprotein-2, % of total | 68.5 | 67.0 | 2.3 | NS |
| Low Density Lipoprotein, % of total | 11.8 | 17.4 | 1.4 | <0.01 |
| Very Low Density Lipoprotein, % of total | 3.98 | 3.19 | 0.70 | NS |
| Chylomicrons, % of total | 0.43 | 0.92 | 0.20 | 0.08 |
| Testosterone, pg/mL | 82.3 | 68.7 | 16.5 | NS |
| Estradiol, pg/mL | 5.65 | 5.22 | 0.21 | NS |
| Cortisol, ug/dL | 4.06 | 4.62 | 0.32 | NS |
| Osteocalcin, ng/mL | 1.76 | 2.03 | 0.35 | NS |
| Amino Terminal Crosslink Telopeptide, nM | 22.8 | 23.3 | 1.4 | NS |
| BCE | | | | |
| Type 2 Cartilage Synthesis, μg/mL | 617.6 | 742.7 | 32.5 | <0.01 |
| Cartilage Oligomeric Matrix Protein, U/L | 2.13 | 2.26 | 0.09 | NS |

*NS = Not significant and P > 0.10

TABLE 2-2
Average Measurements for Female Lean and Overweight Dogs

| | | Female | | |
|---|---|---|---|---|
| | Female Lean | Overweight | Standard | Lean vs |
| Measurement | (n = 15) | (n = 15) | Error | Overweight* |
| Age, years | 8.42 | 6.91 | 0.91 | NS |
| Body Condition Score | 2.40 | 4.47 | 0.12 | <0.01 |
| Body Weight, kg | 10.76 | 15.29 | 0.60 | <0.01 |
| Glucose, mg/dL | 84.46 | 93.07 | 2.82 | 0.04 |
| Insulin, pmol/L | 59.07 | 106.80 | 19.81 | 0.09 |
| Alanine amino-transferase, U/L | 54.20 | 53.00 | 13.74 | NS |
| Alkaline Phosphatase, U/L | 116.07 | 182.27 | 35.28 | NS |
| Cholesterol, mg/dL | 196.13 | 230.00 | 12.81 | 0.07 |
| Triglycerides, mg/dL | 68.47 | 251.00 | 79.95 | NS |
| Total Bilirubin, mg/dL | 0.23 | 1.09 | 0.68 | NS |
| Total Protein, g/dL | 6.01 | 5.89 | 0.20 | <0.01 |
| Creatinine, mg/dL | 0.68 | 0.57 | 0.03 | 0.01 |
| Serum Urea Nitrogen | 12.89 | 8.92 | 0.67 | <0.01 |
| Albumin:Globulin | 1.31 | 1.24 | 0.08 | NS |
| Albumin, g/dL | 3.35 | 3.69 | 0.08 | <0.01 |
| Thyroxine, ug/dL | 1.71 | 2.25 | 0.15 | 0.01 |
| Thyroid Stimulating Hormone, ng/mL | 0.18 | 0.21 | 0.03 | NS |
| Calcium, mg/dL | 9.85 | 10.53 | 0.19 | 0.01 |
| Phosphorous, mg/dL | 4.34 | 4.49 | 0.21 | NS |
| Chloride, mmol/L | 116.93 | 113.67 | 0.63 | <0.01 |
| Potassium, mmol/L | 4.59 | 4.41 | 0.08 | NS |
| Magnesium, mg/dL | 2.76 | 2.93 | 0.10 | NS |
| Sodium, mmol/L | 158.40 | 157.47 | 0.72 | NS |
| Sodium:Potassium | 34.67 | 25.80 | 0.65 | NS |
| Glucagon Like Protein-1, pM | 8.16 | 15.74 | 3.26 | NS |
| Insulin Like Growth Factor-1, ng/mL | 94.7 | 191.2 | 24.5 | <0.01 |
| Ghrelin, ng/mL | 2.39 | 2.08 | 0.33 | NS |
| Leptin, ng/mL | 1.00 | 4.16 | 0.69 | <0.01 |
| Angiotensin I, ng/mL | 0.60 | 0.66 | 0.07 | NS |
| Angiotensin II, ng/mL | 0.62 | 0.84 | 0.46 | NS |
| C-reactive Protein, ng/mL | 4.87 | 2.89 | 0.88 | NS |
| Non-esterified fatty acids, mM | 0.62 | 0.73 | 0.10 | NS |
| High Density Lipoprotein-1, % of total | 12.0 | 12.2 | 2.3 | NS |
| High Density Lipoprotein-2, % of total | 91.8 | 65.2 | 3.3 | NS |
| Low Density Lipoprotein, % of total | 11.9 | 18.6 | 1.9 | 0.02 |
| Very Low Density Lipoprotein, % of total | 4.03 | 3.05 | 0.99 | NS |
| Chylomicrons, % of total | 0.20 | 0.87 | 0.28 | 0.09 |
| Testosterone, pg/mL | 52.1 | 96.3 | 23.3 | NS |
| Estradiol, pg/mL | 5.72 | 5.13 | 0.30 | NS |
| Cortisol, ug/dL | 3.90 | 4.88 | 0.45 | NS |
| Osteocalcin, ng/mL | 1.53 | 2.00 | 0.49 | NS |
| Amino Terminal Crosslink Telopeptide, nM | 23.5 | 23.9 | 2.0 | NS |
| BCE | | | | |
| Type 2 Cartilage Synthesis, μg/mL | 658.5 | 741.0 | 46.0 | NS |
| Cartilage Oligomeric Matrix Protein, U/L | 2.11 | 2.25 | 0.13 | NS |

*NS = Not significant and P > 0.10

TABLE 2-3
Average Measurements for Male Lean and Overweight Dogs

| | | Male | | |
|---|---|---|---|---|
| | Male Lean | Overweight | Standard | Lean vs |
| Measurement | (n = 15) | (n = 15) | Error | Overweight* |
| Age, years | 8.07 | 6.48 | 0.91 | NS |
| Body Condition Score | 2.67 | 4.93 | 0.12 | <0.01 |
| Body Weight, kg | 11.60 | 19.22 | 0.60 | <0.01 |
| Glucose, mg/dL | 83.73 | 93.33 | 2.82 | 0.02 |
| Insulin, pmol/L | 75.60 | 146.29 | 20.14 | 0.02 |
| Alanine amino-transferase, U/L | 52.60 | 81.13 | 13.74 | NS |
| Alkaline Phosphatase, U/L | 114.5 | 200.33 | 35.88 | 0.10 |
| Cholesterol, mg/dL | 207.73 | 226.73 | 12.81 | NS |
| Triglycerides, mg/dL | 115.27 | 234.53 | 79.95 | NS |
| Total Bilirubin, mg/dL | 0.57 | 1.63 | 0.68 | NS |
| Total Protein, g/dL | 6.54 | 7.05 | 0.20 | 0.07 |
| Creatinine, mg/dL | 0.68 | 0.63 | 0.03 | NS |
| Serum Urea Nitrogen | 12.01 | 10.09 | 0.67 | 0.05 |
| Albumin:Globulin | 1.09 | 1.13 | 0.08 | NS |
| Albumin, g/dL | 3.34 | 3.57 | 0.08 | 0.04 |
| Thyroxine, ug/dL | 1.71 | 1.79 | 0.15 | NS |
| Thyroid Stimulating Hormone, ng/mL | 0.20 | 0.21 | 0.03 | NS |
| Calcium, mg/dL | 10.00 | 10.46 | 0.19 | 0.09 |
| Phosphorous, mg/dL | 4.05 | 4.81 | 0.21 | 0.02 |
| Chloride, mmol/L | 115.60 | 113.33 | 0.63 | 0.01 |
| Potassium, mmol/L | 4.45 | 4.45 | 0.08 | NS |
| Magnesium, mg/dL | 2.73 | 2.72 | 0.10 | NS |
| Sodium, mmol/L | 157.60 | 157.47 | 0.72 | NS |
| Sodium:Potassium | 35.67 | 35.67 | 0.65 | NS |
| Glucagon Like Protein-1, pM | 6.61 | 10.44 | 3.26 | NS |
| Insulin Like Growth Factor-1, ng/mL | 109.3 | 176.1 | 24.5 | 0.06 |
| Ghrelin, ng/mL | 2.81 | 1.99 | 0.33 | 0.08 |
| Leptin, ng/mL | 0.91 | 4.16 | 0.69 | <0.01 |
| Angiotensin I, ng/mL | 0.62 | 0.65 | 0.07 | NS |
| Angiotensin II, ng/mL | 0.71 | 1.61 | 0.46 | NS |
| C-reactive Protein, ng/mL | 6.21 | 2.20 | 0.88 | <0.01 |
| Non-esterified fatty acids, mM | 0.89 | 0.87 | 0.10 | NS |
| High Density Lipoprotein-1, % of total | 18.7 | 12.2 | 2.3 | 0.02 |
| High Density Lipoprotein-2, % of total | 65.1 | 68.8 | 3.3 | NS |
| Low Density Lipoprotein, % of total | 11.6 | 16.3 | 1.9 | 0.10 |
| Very Low Density Lipoprotein, % of total | 3.94 | 3.33 | 0.99 | NS |
| Chylomicrons, % of total | 0.65 | 0.97 | 0.28 | NS |
| Testosterone, pg/mL | 112.5 | 41.2 | 23.7 | 0.04 |
| Estradiol, pg/mL | 5.57 | 5.32 | 0.30 | NS |
| Cortisol, ug/dL | 4.22 | 4.36 | 0.45 | NS |
| Osteocalcin, ng/mL | 1.99 | 2.06 | 0.49 | NS |
| Amino Terminal Crosslink Telopeptide, nM | 22.1 | 22.8 | 2.0 | NS |
| BCE | | | | |
| Type 2 Cartilage Synthesis, μg/mL | 576.7 | 744.4 | 46.0 | 0.01 |
| Cartilage Oligomeric Matrix Protein, U/L | 2.15 | 2.27 | 0.13 | NS |

*NS = Not significant and P > 0.10

Average body condition scores were 4.7 and 2.5 for the overweight and lean groups, respectively. Average body weights were 11.2 and 17.3 kg for the lean and overweight groups, respectively. Serum was analyzed for chemistry screens, obesity markers, thyroid markers and arthritis markers. The overweight group had higher levels of alkaline phosphatase (P=0.04), cholesterol (P=0.04), triglycerides (P=0.06), total protein (P<0.01), albumin (P<0.01), thyroxine (P=0.05), calcium (P<0.01), phosphorous (P=0.04), glucose (P<0.01) insulin (P<0.01), insulin like growth factor-1 (P<0.01), low density lipoprotein (P<0.01), leptin (P<0.01) and type 2 cartilage synthesis (P<0.01) than the lean group. The overweight group had lower levels of creatinine (P 0.01), serum urea nitrogen (P<0.01), chloride (P<0.01) and overweight males had lower levels of testosterone (P=0.04) than the lean group.

Analysis of these detailed biomarker data through stepwise regression indicates that body weight, glucose, sodium, chloride, c-reactive protein and thyroid stimulating hormone are particularly useful parameters for determining body condition score. For example, body condition score may be quantitated by employing the following equation:

$$Body condition score = 3.62352 + ( 0.17443 \times body weight in kg ) + ( 0.01621 \times glucose in mg / dL ) + ( 0.06496 \times sodium in mmol / L ) - ( 0.12439 \times chloride in mmol / L ) - ( 0.05575 \times c \text{-} reactive protein in ng / mL ) + ( 1.72392 \times thyroid stimulating hormone in ng / mL ) .$$

In addition, body condition score may be quantitated using biomarker data that may be obtained in routine veterinary assays. For example, analysis of the levels of biomarkers through stepwise regression indicates that body weight and serum levels of urea nitrogen, sodium and chloride in an animal are particularly useful for determining body condition score. Therefore, body condition score may be determined by applying said data to the following, algorithm:

Body condition score = 3.64120 + (0.18614 × body weight in kg) −
(0.05289 × serum urea nitrogen in mg/dL) + (0.08935 ×
sodium in mmol/L) − (0.14088 × chloride in mmol/L).

### Example 3

### Biomarker Levels in Dogs in Weight loss Study

Twenty dogs are utilized in the weight loss study. The dogs are cared for in accordance with Institutional Animal Care and Use Committee protocols. All dogs begin the study with greater than 37% body fat (of total weight), and remain on the weight loss study for 3 months. Dogs are allotted to one of two treatments (Table 3-1). Each food is kibbled and formulated in accordance with the Association of American Feed Control Officials nutrient guide for dogs and is balanced to meet adult maintenance requirements. All dogs undergo dual-energy x-ray absorptiometry (DXA; DXA-QDR-4500, Hologic, Inc., Waltham, Mass.) scans. Blood sample is pulled at 0, 1, 2 and 3 months. Serum is harvested and stored at −20° C. in 1 ml aliquots. Additionally, dogs are offered enrichment toys, received routine grooming and had daily opportunities for socialization with other dogs and people.

Serum is analyzed for chemistry screens, obesity markers, thyroid markers and arthritis markers. Chemistry screens are preformed at the Hill's Pet Nutrition Center (Topeka, Kans.). Insulin analyses are performed by Michigan State University (Lansing, Mich.). Thyroxine thyroid stimulating hormone, glucagon like protein-1 insulin like growth factor-1 ghrelin, leptin, angiotensin I and II, c-reactive protein, high density lipoprotein 1 and 2, low density lipoprotein, very low density lipoprotein, chylomicron, testosterone, estradiol, cortisol, osteocalcin, amino terminal crosslink protein type 2 cartilage synthesis and cartilage oligomeric matrix protein are performed by MD Biosciences, Inc. (St. Paul, Minn.).

TABLE 3-1
Nutrient composition of dog foods in the weight loss study

| Nutrient, 100% Dry Matter Basis | Food A* | Food B** |
|---|---|---|
| Crude Protein, % | 25.6 | 24.9 |
| Crude Fat, % | 8.6 | 7.9 |
| Crude Fiber, % | 21.4 | 21.1 |
| Ash, % | 5.6 | 5.1 |
| Calcium, % | 0.67 | 0.91 |
| Phosphorous, % | 0.54 | 0.64 |
| Lysine, % | 1.41 | 1.43 |
| Methionine + Cystine, % | 0.73 | 0.79 |
| Tryptophan, % | 0.29 | 0.24 |
| Threonine, % | 0.91 | 0.90 |
| Arginine, % | 1.43 | 1.53 |
| Isoleucine, % | 0.83 | 1.05 |
| Valine, % | 1.23 | 1.26 |
| Leucine, % | 1.81 | 2.03 |
| Histidine, % | 0.72 | 0.57 |
| Phenylalanine + Tyrosine, % | 1.70 | 1.64 |
| Carnitine, ppm | 300 | 300 |
| Metabolizable Energy, kcal/kg | 2992 | 2966 |

*Food A = Hill's ® Canine Prescription Diet ® r/d ® Canned. Ingredients: Water, pork by-products, soybean mill run, rice, pork liver, powdered cellulose, soybean meal, chicken liver flavor, vegetable oil, iron oxide, taurine, L-carnitine, minerals (calcium carbonate, dicalcium phosphate, salt, zinc oxide, ferrous sulfate, copper sulfate, manganous oxide, calcium iodate, sodium selenite), beta-carotene, vitamins (choline chloride, vitamin D3 supplement, vitamin E supplement, ascorbic acid, thiamine mononitrate, niacin, calcium pantothenate, pyridoxine hydrochloride, riboflavin, folic acid, biotin, vitamin B12 supplement).
**Food B = Hill's ® Canine Prescription Diet ® r/d ® Dry. Ingredients: Corn meal, peanut hulls 28.2% (a source of fiber), chicken by-product meal, soybean meal, soybean mill run, chicken liver flavor, dried egg product, vegetable oil, taurine, L-carnitine, preserved with BHT, BHA and ethoxyquin, minerals (salt, ferrous sulfate, zinc oxide, copper sulfate, manganous oxide, calcium iodate, sodium selenite), beta-carotene, vitamins (choline chloride, vitamin A supplement, vitamin D3 supplement, vitamin E supplement, L-ascorbyl-2-polyphosphate (a source of vitamin C), niacin, thiamine mononitrate, calcium pantothenate, pyridoxine hydrochloride, riboflavin, folic acid, biotin, vitamin B12 supplement).

Results indicate that dogs fed Food A had significant weight loss (−4924 g; P<0.01) lean loss (−721 g: P<0.01) and fat loss (−4167 g; P<0.01) at day 90 when compared to day 0. Dogs fed Food B had significant weight loss (−3466 g; P<0.01) and fat loss (−3363 g; P<0.01) at day 90 when compared to day 0. No differences were observed for lean when dogs were fed Food B. See Table 3-2.

Serum chemistry screens and electrolytes are presented in Table 3-3 Dogs fed Food A had a decrease in globulin (P<0.01), total protein (P<0.01), alkaline phosphatase (P=0.03), alanine amino transferase (P=0.02), albumin (P<0.01), cholesterol (P<0.01), triglycerides (P<0.01), phosphorus (P<0.01), sodium (P<0.01), sodium: potassium (P=0.02) and an increase in calcium (P=0.02), potassium (P=0.05) and chloride (P<0.01). Dogs fed Food B had a decrease in albumin (P<0.01), total protein (P<0.01), cholesterol (P<0.01), sodium (P<0.01), sodium: potassium (P<0.01) and an increase in calcium (P<0.01), chloride (P<0.01), potassium (P<0.01), magnesium (P<0.01) and serum urea nitrogen (P=0.03). See Table 3-2 and 3-3 below.

TABLE 3-2
Body composition of dogs after consuming weight loss foods

| Body Parameter Measured | Food A | Food B |
|---|---|---|
| Weight day 0, g | 17569 | 17257 |
| Weight day 30, g | 15394 | 15798 |
| Weight day 60, g | 13970 | 14715 |
| Weight day 90, g | 12645 | 13791 |
| Weight change day 0 to 30, g | −2304 | −1459 |
| Weight change day 0 to 60, g | −3728 | −2542 |
| Weight change day 0 to 90, g | −4924 | −3466 |
| Day 0 vs day 30* | <0.01 | <0.01 |
| Day 0 vs day 60* | <0.01 | <0.01 |
| Day 0 vs day 90* | <0.01 | <0.01 |
| Lean day 0, g | 9678 | 9434 |
| Lean day 30, g | 9093 | 9295 |
| Lean day 60, g | 9059 | 9303 |
| Lean day 90, g | 8961 | 9367 |
| Lean change day 0 to 30, g | −585 | −139 |
| Lean change day 0 to 60, g | −619 | −131 |
| Lean change day 0 to 90, g | −721 | −67 |
| Day 0 vs Day 30* | <0.01 | NS |
| Day 0 vs day 60* | <0.01 | NS |
| Day 0 vs day 90* | <0.01 | NS |
| Fat day 0, g | 7411 | 7343 |
| Fat day 30, g | 5830 | 6028 |
| Fat day 60, g | 4455 | 4952 |
| Fat day 90, g | 3244 | 3979 |
| Fat change day 0 to 30, g | −1705 | −1314 |
| Fat change day 0 to 60, g | −3081 | −2390 |
| Fat change day 0 to 90, g | −4167 | −3363 |
| Day 0 vs day 30* | <0.01 | <0.01 |
| Day 0 vs day 60* | <0.01 | <0.01 |
| Day 0 vs day 90* | <0.01 | <0.01 |

*Probability of greater F-value

TABLE 3-3
Blood chemistry screens and markers of dogs after consuming weight loss
foods for 90 days

| | Food A | | | | Food B | | | |
|---|---|---|---|---|---|---|---|---|
| | | Day | | Day 0 vs | | Day | | Day 0 vs |
| Analyte | Day 0 | 90 | Change | Day 90 | Day 0 | 90 | Change | Day 90 |
| Albumin g/dL | 3.60 | 3.33 | −0.27 | <0.01 | 3.54 | 3.29 | −0.25 | <0.01 |
| Serum Urea | 12.8 | 13.0 | 0.2 | NS | 8.8 | 12.5 | 3.7 | 0.03 |
| Nitrogen, mg/dL | | | | | | | | |
| Creatinine, mg/dL | 0.60 | 0.64 | 0.04 | 0.09 | 0.59 | 0.63 | 0.04 | NS |
| Total Protein, g/dL | 6.21 | 5.57 | −0.64 | <0.01 | 0.65 | 0.60 | −0.05 | NS |
| Alkaline | 307 | 92 | −216 | 0.03 | 175 | 100 | −76 | NS |
| Phosphatase, U/L | | | | | | | | |
| Cholesterol, mg/dL | 229 | 166 | −64 | <0.01 | 222 | 169 | −53 | <0.01 |
| Glucose, mg/dL | 106 | 99 | −7 | NS | 101 | 96 | −5 | NS |
| Insulin, IU/mL | 10.29 | 3.08 | 6.86 | <0.01 | 5.50 | 3.47 | 2.25 | NS |
| Triglycerides, | 191 | 136 | −55 | <0.01 | 147 | 138 | −10 | NS |
| mg/dL | | | | | | | | |
| Calcium, mg/dL | 9.8 | 10.2 | 0.4 | 0.02 | 9.7 | 10.2 | 0.5 | <0.01 |
| Chloride, mg/dL | 114 | 120 | 6 | <0.01 | 115 | 120 | 5 | <0.01 |
| Phosphorus, mg/dL | 4.42 | 3.28 | −1.14 | <0.01 | 3.43 | 3.18 | −0.25 | NS |
| Ghrelin, ng/mL | 1.69 | 2.19 | 0.50 | 0.02 | 2.38 | 2.38 | 0.01 | NS |
| Leptin, ng/mL | 3.00 | 0.20 | −2.80 | <0.01 | 1.89 | 0.29 | −1.60 | <0.01 |
| Amino Terminal | 19.1 | 20.7 | 1.6 | NS | 22.3 | 22.1 | −0.2 | NS |
| Crosslink | | | | | | | | |
| Telopeptide, nM | | | | | | | | |
| BCE | | | | | | | | |
| Type 2 Cartilage | 1179 | 1099 | −80 | NS | 1007 | 1049 | 42 | NS |
| Synthesis, μg/mL | | | | | | | | |
| Cartilage | 1.73 | 1.38 | −0.35 | <0.01 | 1.72 | 1.53 | −0.19 | NS |
| Oligomeric Matrix | | | | | | | | |
| Protein, U/L | | | | | | | | |
| C-reactive Protein, | 2.79 | 2.26 | −0.53 | NS | 2.10 | 1.25 | −0.76 | NS |
| ng/mL | | | | | | | | |

An objective of the studies disclosed herein is to determine what biomarkers differ between lean and overweight dogs. By identifying differences in biological markers between lean and overweight animals, veterinarians can not only definitively quantitate a body condition score, but can also diagnose body weight condition or predisposition thereto as well as diagnose an obesity-related health disorder or predisposition thereto. These markers could be utilized by the veterinarian to manage weight loss regimens with blood analysis along with body weight reduction.

As described in Example 2, the overweight group had elevated levels of glucose, insulin, insulin like growth factor-1 and glucagon like protein-1 suggesting the signs of insulin resistance. The results are not surprising because diabetes and insulin resistance are commonly associated with obesity. The data in Example 3 indicate dogs going through weight loss had a reduction in glucose and insulin indicating that weight loss can correct the obesity related glucose disorders.

The overweight dogs had elevated levels of triglycerides, cholesterol, low density lipoprotein, chylomicrons and lowered levels of high density lipoprotein-1 are common signs of dyslipidemia. Studies with dogs have demonstrated that dyslipidemia is often associated with insulin resistance. Insulin resistance plays a central role in the development of hyperlipidemia. The increase in blood triglyceride concentration results from the increase in the production of triglyceride rich lipoproteins and a decrease in their catabolism. Abnormalities in insulin action can result from an increase in lipolysis in adipocytes which results in increased tatty acid release and repackaging, of the fatty acids back into triglycerides at the liver.

In Example 3, dogs on a weight loss regime had a reduction in cholesterol and triglycerides indicating that the signs of dyslipidemia can be corrected through food and weight loss. This is consistent with other published canine weight loss studies. Diez at al., “Evolution of blood parameters during weight loss in experimental obese beagle dogs” J. Anim. Physiol. a. Anim. Nutr. 2004; 88:166-171, fed overweight beagles either a high protein (47.5% protein and 10.9% crude fiber) or a high fiber (23.8% protein and 23.3% crude fiber) diet during their weight program. They observed decreases in both triglycerides and cholesterol when dogs were fed either of the two weight loss foods, indicating that these observed changes were not diet related but were directly related to weight loss. The observed decrease in triglycerides and cholesterol resulting from weight loss in the current study and Diez et al. is something that can be measured by the veterinarian during routine chemistry screens. Measuring cholesterol and triglycerides for obesity issues and monitoring weight loss may be a way to discuss the importance of obesity without offending the pet owner. It is important to note that in the study by Diez et al. and the current study, elevated triglycerides and cholesterol are both within the normal published ranges for the dog. Thus, looking for abnormally high triglyceride and cholesterol values may not be a good indicator for obesity and more focus should be given to elevated levels within normal ranges.

The overweight also group had increased levels of arthritic markers, even though they did not show any signs of arthritis (i.e. lameness). Although all arthritic markers were elevated in the overweight group, only alkaline phosphatase and type II cartilage synthesis were statistically significant. The increase in both alkaline phosphatase and type II cartilage synthesis could be an early indicator of osteoarthritis in overweight dogs. Alkaline phosphatase is typically elevated when dogs have bone, bile duct and/or liver disorders. The elevated alkaline phosphatase in this study is likely associated with bone because alanine amino-transferase did not differ between the two groups and albumin was higher in the overweight group thus ruling out any potential Liver disorders.

Type II cartilage synthesis typically increases when cartilage damage occurs. The cartilage matrix consists of two major components, type II cartilage and the proteoglycan aggrecan. Cartilage fibrils provide tensile strength to maintain tissue integrity. Aggrecan is interwoven with the cartilage fibrils and contributes to cartilage matrix compressive stiffness. Damage to type II cartilage and loss of aggrecan are fundamental features of damage to articular cartilage in osteoarthritis. This damage has been linked to proteolytic enzymes secreted by chondrocytes and synoviocytes. The matrix metalloproteinase family (i.e. MMP-13) is responsible for the primary cleavage of the triple helix of type II cartilage. In Example 3, dogs going through weight loss had a decrease in alkaline phosphatase indicating that managing obesity may help/prevent the onset of arthritis. Because these foods do not have added joint benefits for reducing/treating arthritis (i.e. n−3 fatty acids, glucosamine and/or chondroitin), it becomes apparent that weight loss alone lowered the alkaline phosphatase levels. This is likely the result of reducing the load exerted on the joints when animals reduce the body weight. Thus, it is further contemplated herein that levels of arthritic biomarker, e.g., alkaline phosphatase and type II cartilage synthesis, may be used as biomarker to predict a predisposition to osteoarthritis in an animal.

Leptin, ghrelin and GLP-1 concentrations were also measured in both Examples 2 and 3 because of their known effects on appetite suppression and stimulation. The overweight group had elevated levels of leptin along with lower levels of ghrelin. These results are in agreement with Jeusette et al., “Influence of obesity on plasma lipid and lipoprotein concentrations in dogs” Am. J. Vet. Res. 2005; 66:81-86, and Sagawa et al., “Correlation between plasma leptin concentration and body fat content in dogs” Am. J Vet. Res. 2002; 63(1):7-10. In both studies leptin concentrations were directly related to body fat mass in overweight beagles. In the current study, leptin concentrations decreased with decreasing fat mass during weight loss. Jeusette et al. also observed a decrease in ghrelin concentrations in overweight dogs and believed to be the result of ghrelin being down regulated resulting from excess energy storage. As disclosed herein, it appears that ghrelin levels are not affected by weight loss when dogs are fed the dry food. However, ghrelin levels did increase when dogs are fed the canned food for weight loss. This may be the result of increased gut fill from higher intakes of the canned product. GLP-1 also plays a role in the control of nutrients flowing from the stomach to the small intestine through its inhibitory effects on gastrointestinal transit and gastric emptying. This GLP-1 mechanism is believed to exert its effect on appetite. The overweight dogs had increased levels of GLP-1 when compared to the lean group. These results indicate that these hormones may be trying to reduce intake in the overweight dog group; however, their effects on intake are not being elicited.

The results of these studies indicate that obesity is directly related to other disease states in dogs. The markers in Example 2 indicate that overweight dogs showed early signs of dyslipidemia, arthritis and diabetes, Example 3 demonstrates that many of these differences can be alleviated through weight loss.

### Example 4

### Prediction of Body Weight Condition in Cats

Thirty lean and thirty overweight eats were identified for this study. Cats with a body condition score (BCS) of 4 or 5 were classified as obese/overweight for purposes of this study (on a scale of 1 through 5 where 1 equals thin and 5 equals obese). Cats with a BCS less than 3 were classified as lean. Animals were weighed, given a body condition score and a blood sample was drawn. Serum was harvested and stored at −20° C. in 1 mL aliquots.

Serum was analyzed for chemistry screens, obesity markers, thyroid markers and arthritis markers. Chemistry screens were preformed at the Hill's Pet Nutrition Center (Topeka, Kans.). Insulin analysis was performed by Michigan State University (Lansing, Ma). Thyroxine, thyroid stimulating hormone, ghrelin, leptin, angiotensin I and II, osteocalcin, amino terminal crosslink protein, bone-specific alkaline phosphatase and carboxy terminal crosslink telopeptide were performed by MD Biosciences, Inc. (St. Paul, Minn.).

Data were analyzed using General Linear Models procedure of SAS (1989) to determine treatment means. The experimental unit was cat. Differences were considered significant when P<0.05 and trends were determined when P<0.10.

TABLE 4-1
Average Measurements for Lean and Obese Cats

| | | Male | | |
|---|---|---|---|---|
| | Male Lean | Overweight | Standard | Lean vs |
| Measurement | (n = 30) | (n = 30) | Error | Overweight* |
| Age, years | 6.43 | 8.43 | 0.53 | 0.01 |
| Body Condition Score | 2.48 | 4.23 | 0.09 | <0.01 |
| Body Weight, kg | 3.22 | 5.83 | 0.17 | <0.01 |
| General Metabolism Markers | | | | |
| Glucose, mg/dL | 79.4 | 87.0 | 2.3 | 0.02 |
| Insulin, pmol/L | 17.4 | 13.7 | 2.3 | NS |
| Organ Function Markers | | | | |
| Alanine amino-transferase, U/L | 51.7 | 51.6 | 2.5 | NS |
| Alkaline Phosphatase, U/L | 35.0 | 42.0 | 2.7 | 0.07 |
| Cholesterol, mg/dL | 168.7 | 174.5 | 7.9 | NS |
| Triglycerides, mg/dL | 38.8 | 56.3 | 6.1 | 0.05 |
| Total Bilirubin, mg/dL | 0.22 | 0.21 | 0.03 | NS |
| Total Protein, g/dL | 7.38 | 7.78 | 0.10 | <0.01 |
| Creatinine, mg/dL | 1.20 | 1.19 | 0.06 | NS |
| Serum Urea Nitrogen, mg/dL | 22.3 | 23.1 | 0.8 | NS |
| Serum Urea Nitrogen:Creatinine | 19.2 | 20.1 | 0.7 | NS |
| Albumin:Globulin | 0.76 | 0.79 | 0.03 | NS |
| Albumin, g/dL | 3.15 | 3.40 | 0.06 | <0.01 |
| Globulin, g/dL | 4.24 | 4.37 | 0.11 | NS |
| Thyroxine, ug/dL | 2.51 | 2.77 | 0.08 | 0.02 |
| Thyroid Stimulating Hormone, ng/mL | 0.08 | 0.05 | 0.01 | 0.02 |
| Electrolytes | | | | |
| Calcium, mg/dL | 9.48 | 9.82 | 0.14 | 0.09 |
| Phosphorous, mg/dL | 4.87 | 4.46 | 0.16 | 0.08 |
| Chloride, mmol/L | 122.2 | 122.6 | 0.5 | NS |
| Potassium, mmol/L | 4.48 | 4.7 | 0.07 | 0.03 |
| Magnesium, mg/dL | 2.38 | 2.84 | 0.07 | <0.01 |
| Sodium, mmol/L | 165.7 | 164.5 | 0.5 | NS |
| Sodium:Potassium | 37.1 | 35.2 | 0.5 | 0.02 |
| Obesity Markers | | | | |
| Ghrelin, ng/mL | 1.90 | 1.63 | 0.10 | 0.06 |
| Leptin, ng/mL | 4.28 | 45.30 | 2.75 | <0.01 |
| Angiotensin I, ng/mL | 7.79 | 2.38 | 1.13 | NS |
| Angiotensin II, ng/mL | 1.06 | 1.59 | 0.36 | NS |
| Arthritis and Bone Markers | | | | |
| Osteocalcin, ng/mL | 0.70 | 0.47 | 0.12 | NS |
| Amino Terminal Crosslink Telopeptide, nM | 22.5 | 19.7 | 1.7 | NS |
| BCE | | | | |
| Bone-Specific Alkaline Phaosphatase, ng/mL | 8.76 | 7.85 | 0.78 | NS |
| Carboxy Terminal Crosslink Telopeptide, μg/L | 9.62 | 8.21 | 0.82 | NS |

*NS = Not significant and P > 0.10

Average body condition scores were 4.2 and 2.5 for the overweight and lean groups, respectively. Average body weights were 5.8±0.2 and 3.2±0.2 kg for the overweight and lean groups, respectively. Serum was analyzed for chemistry screens obesity markers, thyroid markers and arthritis markers. The overweight group had higher levels of alkaline phosphatase (P=0.07), triglycerides (P=0.05), total protein (P<0.01), albumin (P<0.01) potassium (P=0.03), magnesium (P<0.01), sodium: potassium (P=0.02), glucose (P=0.02), leptin (P<0.01) and thyroxine (P=0.02). The overweight group had lower levels of thyroid stimulating hormone (P=0.02) and ghrelin (P 0.06).

Analysis of these detailed biomarker data through stepwise regression indicates that body weight and serum levels of sodium potassium, chloride, phosphorus, bilirubin and ghrelin are particularly useful parameters for quantitating body condition score (i.e. body weight condition) in cats. For example, body condition score may be quantitated by employing the following equation:

Body condition score = −3.20078 + (0.4259 × body weight in kg) −
(0.05508 × sodium in mmol/L) + (0.69884 × potassium in mmol/L) +
(0.09472 × chloride in mmol/L) − (0.15372 × phosphorus in mg/dL) +
(1.31580 × total bilirubin in mg/dL) − (0.35136 × ghrelin in ng/mL).

In addition, body condition score may be quantitated using biomarker data that may be obtained in routine veterinary assays. For example, analysis of the levels of biomarkers through stepwise regression indicates that body weight and serum levels of blood urea nitrogen creatinine, potassium, chloride, phosphorus and bilirubin are particularly useful for determining body condition score in cats. Therefore, body condition score may be determined by applying said data to the following algorithm:

Body condition score = −7.34191 + (0.48335 × body weight in kg) +
(0.03578 × blood urea nitrogen:creatinine) + (0.58860 ×
potassium in mmol/L) + (0.04683 × chloride in mmol/L) − (0.16894 ×
phosphorus in mg/dL) + (0.86613 × total bilirubin in mg/dL).

In the specification, there have been disclosed typical preferred embodiments of the invention and, although specific terms are employed, they are used in a generic and descriptive sense only and not for purposes of limitation, the scope of the invention being set forth in the claims. Obviously many modifications and variations of the invention are possible in light of the above teachings. It is therefore to be understood that within the scope of the appended claims the invention may be practiced otherwise than as specifically described.

What is claimed is:

1. A method for diagnosing a body weight condition or predisposition to a body weight condition in an animal comprising determining observed level(s) of at least one biomarker in a tissue or biofluid sample from the animal and comparing the observed level(s) to reference level(s) for the biomarker; wherein the observed level(s) relative to the reference level(s) are individually or collectively indicative of the body weight condition or predisposition.

2. The method of claim 1 wherein the animal is canine or feline.

3. The method of claim 1 wherein the animal is up to about one year of age and the observed level(s) relative to the reference level(s) are individually or collectively indicative of predisposition to a body weight condition later in the animal's life.

4. The method of claim 1 wherein the condition or predisposition is obesity or a propensity to gain weight.

5. The method of claim 1 wherein the condition or predisposition increases the animals risk for an obesity-related health disorder.

6. The method of claim 5 wherein the obesity-related heath disorder is selected from the group consisting of hyperlipidemia, dyslipidemia, insulin resistance, glucose intolerance, hepatic lipidosis, anesthetic complications, hyperadrenocorticism, hypothyroidism, diabetes mellitus, insulinoma, pituitary chromophobe adenoma, hypopituitarism, hypothalamic lesions, joint stress, musculoskeletal pain, dyspnea, hypertension, dystocia, exercise intolerance, heat intolerance, decreased immune function, degenerative joint and orthopedic diseases, cardiovascular diseases, hypertension, respiratory distress, altered kidney function, pancreatitis, transitional cell carcinomas, fatigue sleep disorders, reproductive disorders, and combinations thereof.

7. The method of claim 1 wherein the biomarker is selected from the group consisting of glucose, GLP-1, ghrelin c-reactive protein, thyroid stimulating hormone, and combinations thereof.

8. The method of claim 1 wherein observed level(s) are determined for at least two biomarkers.

9. The method of claim 1 wherein the tissue or biofluid sample is obtained when the animal is in a fasted state.

10. The method of claim 1 wherein tissue or biofluid samples are obtained at a plurality of time points during a feeding cycle, including at least one preprandial time point and at least one postprandial time point.

11. The method of claim 1 wherein the tissue or biofluid is whole blood, blood plasma or blood serum.

12. The method of claim 1 wherein the observed and reference levels are determined using one or more assays independently selected from the group consisting of enzyme immunoassays, enzyme-linked immunosorbent assays, immunofluorescent assays, radioimmunoassays, western blot assays, biochemical assays, enzymatic assays, and colorimetric assays.

13. The method of claim 1 wherein (a) the animal is canine, (b) the biomarker comprises glucose in serum, and (c) when the observed body weight-adjusted serum glucose level in a fasted animal is at least about 10% lower than the body weight-adjusted reference level for a canine of normal weight, a predisposition of the animal to gain weight is diagnosed.

14. The method of claim 1 wherein (a) the animal is canine, (b) the biomarker comprises GLP-1 in serum, and (c) when the observed body weight-adjusted serum GLP-1 level in a fasted animal is at least about 20% lower than the body weight-adjusted reference level for a canine of normal weight, a predisposition of the animal to gain weight is diagnosed.

15. The method of claim 1 wherein (a) the animal is canine, (b) the biomarker comprises ghrelin in serum, and (c) when the observed body weight-adjusted serum ghrelin level in a fasted animal is at least about 20% lower than the body weight-adjusted reference level for a canine of normal weight, a predisposition of the animal to gain weight is diagnosed.

16. A method for selecting a regimen for an animal comprising (a) diagnosing a body weight condition or predisposition by determining observed level(s) of at least one biomarker in a tissue or biofluid sample from the animal, and comparing the observed level(s) to reference level(s) for the biomarker; wherein the observed level(s) relative to the reference level(s) are individually or collectively indicative of the body weight condition or predisposition; and (b) identifying a regimen appropriate to the body weight condition or predisposition diagnosed.

17. The method of claim 16 wherein the regimen comprises a composition for consumption by the animal.

18. The method of claim 16 wherein the regimen comprises a form of exercise for the animal.

19. A method for detecting onset of a body weight condition or predisposition in an animal comprising monitoring at least one biomarker in the animal over a period by determining, at each of a plurality of time points during the period, observed level(s) of the biomarker in a tissue or biofluid sample from the animal, and comparing the observed level(s) to reference level(s) for the biomarker; wherein onset is detected if, at any time point, the observed level(s) relative to the reference level(s) are individually or collectively indicative of the body weight condition or predisposition.

20. The method of claim 19 further comprising monitoring the animal's body weight by a technique selected from the group consisting of weighing, assessment of relative body weight, assessment of body condition score, morphometry, and combinations thereof.

21. A method for assessing the efficacy of a regimen for managing a body weight condition or predisposition in an animal comprising monitoring at least one biomarker in the animal over a period during which the regimen is administered, by determining, at each of a plurality of time points during the period, observed level(s) of the biomarker in a tissue or biofluid sample from the animal, and comparing the observed level(s) to reference level(s) for the biomarker; wherein the observed level(s) relative to the reference level(s) are individually or collectively indicative of the efficacy of the regimen in managing the body weight condition or predisposition.

22. A kit comprising
(a) one or more reagents for detecting observed level(s) of at least one biomarker in a tissue or biofluid sample from an animal; and
(b) one or more user-accessible media carrying information that comprises (i) reference level(s) of the biomarker; and (ii) an algorithm that compares the observed level(s) to the reference level(s);
wherein the observed level(s) relative to the reference level(s) are individually or collectively indicative of a body weight condition or predisposition in the animal.

23. The kit of claim 22 wherein the one or more reagents comprise at least one reporter moiety or label.

24. The kit of claim 22 wherein the one or more reagents comprise at least one antibody.

25. The kit of claim 22 further comprising means for communicating information that comprises one or more of (a) a diagnosis of a body weight condition or predisposition as indicated by the observed level(s) relative to the reference level(s); and (b) a suggested or prescribed regimen appropriate to the diagnosis.

26. A method for diagnosing a predisposition for an obesity-related health disorder in an animal comprising determining observed level(s) of at least one biomarker in a tissue or biofluid sample from the animal and comparing the observed level(s) to reference level(s) for the biomarker; wherein the observed level(s) relative to the reference level(s) are individually or collectively indicative said predisposition.

27. The method of claim 26 wherein the animal is canine or feline.

28. The method of claim 26 wherein the animal is up to about one year of age and the observed level(s) relative to the reference level(s) are individually or collectively indicative of predisposition to a body weight condition later in the animal's life.

29. The method of claim 26 wherein the obesity-related heath disorder is selected from the group consisting of hyperlipidemia, dyslipidemia, insulin resistance, glucose intolerance, hepatic lipidosis, anesthetic complications, hyperadrenocorticism, hypothyroidism, diabetes mellitus, insulinoma, pituitary chromophobe adenoma, hypopituitarism, hypothalamic lesions, joint stress, musculoskeletal pain, dyspnea, hypertension, dystocia, exercise intolerance, heat intolerance, decreased immune function, degenerative joint and orthopedic diseases, cardiovascular diseases, hypertension, respiratory distress, altered kidney function, pancreatitis, transitional cell carcinomas, fatigue, sleep disorders, reproductive disorders, and combinations thereof.

30. The method of claim 26 wherein the biomarker is selected from the group consisting of glucose, GLP-1, ghrelin, c-reactive protein, thyroid stimulating hormone, and combinations thereof.

31. The method of claim 26 wherein observed level(s) are determined for at least two biomarkers.

32. The method of claim 26 wherein the tissue or biofluid sample is obtained when the animal is in a fasted state.

33. The method of claim 26 wherein tissue or biofluid samples are obtained at a plurality of time points during a feeding cycle, including at least one preprandial time point and at least one postprandial time point.

34. The method of claim 26 wherein the tissue or biofluid is whole blood, blood plasma or blood serum.

35. The method of claim 26 wherein the observed and reference levels are determined using one or more assays independently selected from the group consisting of enzyme immunoassays, enzyme-linked immunosorbent assays, immunofluorescent assays, radioimmunoassays, western blot assays, biochemical assays, enzymatic assays, and colorimetric assays.

36. The method of claim 26, wherein said overweight-related health disorder is osteoarthritis

37. The method of claim 26, wherein said biomarker is alkaline phosphatase.

38. A method of quantitating a body condition score of an animal comprising (a) analyzing the body weight and serum levels of at least one biomarker in said animal; and (b) applying said data obtained from step (a) to any of Algorithm I-IV of the invention.
